(19) United States
(12) Patent Application Publication
(10) Pub. No.: US 2023/0056701 A1
(43) Pub. Date: Feb. 23, 2023

(54) DNA AMPLIFICATION METHOD, DNA AMPLIFICATION KIT, AND PROFILING/DIAGNOSIS METHOD

(71) Applicant: NEC Corporation, Minato-ku, Tokyo (JP)

(72) Inventors: Minoru ASOGAWA, Tokyo (JP); Asumi INAGUMA, Tokyo (JP)

(73) Assignee: NEC Corporation, Minato-ku, Tokyo (JP)

(21) Appl. No.: 17/777,346

(22) PCT Filed: Nov. 22, 2019
(86) PCT No.: PCT/JP2019/045846

§ 371 Date: May 17, 2022

(51) Int. Cl. C12Q 1/6886

(52) U.S. Cl. CPC C12Q 1/6886; C12Q 1/6858

(57) ABSTRACT

There is provided a DNA amplification method, comprising: preparation of a reaction solution containing a template DNA comprising a microsatellite, primers, a polymerase, and a recombinase, and subjection of the reaction solution to constant temperature incubation so as to amplify a DNA sequence comprising the microsatellite in the template DNA.

### FIELD

The disclosure relates to a DNA amplification method, a DNA amplification kit and a profiling/diagnosis method, particularly, to a DNA amplification method and a DNA amplification kit for amplifying a DNA sequence comprising a microsatellite, and a profiling/diagnosis method of executing DNA profiling and/or cancer diagnosis using the DNA amplification method.

### BACKGROUND

There are technologies for executing DNA (deoxyribonucleic acid) profiling and cancer diagnosis based on the number of repeat sequences comprised in a microsatellite. In an ordinary DNA profiling and cancer diagnosis where amplification of a sample DNA sequence by PCR (Polymerase Chain Reaction) is executed, an artifact referred to as stutter generated during the amplification provides a negative effect on accuracy in the profiling and the diagnosis. For example, Patent Literature (PTL) 1 discloses a technology in which a possible level of the maximum stutter peak is estimated and peaks having a lower level are interpreted as the stutter peak so as to eliminate the negative effect due to the stutter.

### CITATION LIST

### Patent Literature

PTL 1: Tokkai JP 2006-163720A

### SUMMARY

### Technical Problem

The following analysis is made from an aspect of the disclosure. Herein, disclosure of the document cited in the Citation List is incorporated into the application by reference thereto.

PTL 1 is a technology involving generation of the stutter as a premise. That is, the technology of PTL 1 would be unnecessary, if the generation of the stutter is prevented or suppressed, since the negative effect along with the generation of the stutter would be fundamentally eliminated. Therefore, there is a demand for provision of a technology in which the generation of the stutter is prevented or suppressed. In other words, it is a purpose of the disclosure to provide a technology for preventing or suppressing the generation of the stutter so as to contribute to improvement in accuracy of DNA profiling and/or cancer diagnosis.

### Solution to Problem

According to a first aspect of the disclosure, there is provided a DNA amplification method, comprising:

a step of preparing a reaction solution containing a template DNA comprising a microsatellite, primers, a polymerase, and a recombinase, and

a step of subjecting the reaction solution to constant temperature incubation so as to amplify a DNA sequence comprising the microsatellite in the template DNA.

According to a second aspect of the disclosure, there is provided a DNA amplification kit, comprising:

a polymerase and a recombinase which are prepared in a reaction solution for amplifying a DNA sequence comprising a microsatellite, and

primers for amplifying the DNA sequence.

According to a third aspect of the disclosure, there is provided a profiling/diagnosis method, comprising:

a step of amplifying a DNA sequence comprising a microsatellite according to the DNA amplification method of the first aspect,

a step of subjecting a reaction solution after an amplification reaction to electrophoresis and/or DNA sequence so as to analyze repeat number of a repeat sequence, and

a step of executing DNA profiling and/or cancer diagnosis using an analysis result by analysis.

### Advantageous Effects of Invention

According to each of the aspects of the disclosure, there are provided a DNA amplification method, a DNA amplification kit, and a profiling/diagnosis method of executing DNA profiling and/or cancer diagnosis using the DNA amplification method, in which generation of stutter is prevented or suppressed so as to contribute to improvement in accuracy of DNA profiling and/or cancer diagnosis.

### BRIEF DESCRIPTION OF THE DRAWINGS

FIGS. 1A and 1B are an explanatory view for explanation of differences between DNA amplification by PCR and the DNA amplification of the disclosure, respectively.

FIG. 2 is a model relating to stutter.

FIGS. 3A to 3D show a model relating to a generation mechanism of stutter.

FIG. 4 is a model relating to DNA amplification by RPA.

FIG. 5 is a flowchart diagram indicating a process flow in the profiling/diagnosis method of the disclosure.

FIG. 6A is DNA amplification result by PCR only, and FIG. 6B is DNA amplification result in combination of PCR and RPA.

### MODES

A possible preferable example embodiment in the disclosure is explained in detail while referring to drawings. Herein, the disclosure is not limited to the following examples and a modification, a variation, an application (including partial one) and a combination thereof can be made as long as not departing from technical concept(s) of the disclosure that can be found from the following examples.

First, terms used in the application are explained.

A term “microsatellite” refers to a repeat sequence itself and a region, position (locus, site) comprising the repeat sequence, and also refers to a name of a locus in the application. Generally, the microsatellite is also referred to as STR (Short Tandem Repeat) sequence. As one example, “D1S1656” and “CSF1PO” in which isoalleles are determined upon DNA profiling are also included in the term “microsatellite”. Herein, D1S1656 is known as having a repeat sequence represented by [TAGA]ₙ [TGA]₀₋₁ [TAGA]ₙ [TAGG]₀₋₁ [TG]₅ (see https://strbase.NIST.gov/str_D1S1656.htm, STRBase of NIST (National Institute of Standards and Technology), and the like). For example, an isoallele of D1S1656 referred to as “15.3 type” has a repeat sequence represented by [TAGA]₄ TGA [TAGA]₁₀ TAGG [TG]₅. Herein, there is also a case where [TG]s is not included in the repeat sequence.

A term “template DNA” refers to DNA to be an initial template of DNA amplification, particularly refers to DNA included in a target sample of DNA profiling and/or a sample obtained from a subject to cancer diagnosis. That is, when executing DNA profiling for a paternity test and the like, cells are obtained from a subject using a swab and the like, and DNA included in such cells corresponds to the template DNA. Also, when executing DNA profiling with a purpose of specifying an offender from bloodstain left in a crime scene, DNA included in bloodstain, body fluid, secretion or residue thereof corresponds to the template DNA. Also, when executing cancer diagnosis, DNA included in cells obtained from a subject corresponds to the template DNA. Further, the cancer diagnosis also includes MSI (microsatellite instability) test (see, for example, https://www.promega.co.jp/msi_analysis/ of Promega Corporation, and the like).

That is, the template DNA may be a target sample of DNA profiling and/or a sample obtained from a subject to cancer diagnosis. In addition, the target sample of DNA profiling may be a sample containing DNAs from a plurality of persons. In addition, the target sample of DNA profiling may be a sample containing DNAs of a plurality of persons at different ratios (i.e., bloodstain). Herein, the template DNA may be one which has been previously prepared. For example, in a purpose of preparing an indicator (i.e., a control, standard) for determination of the repeat number, a sample (amplicon) of which the repeat number has been previously determined is used as the template DNA. In addition, cDNA (complementary DNA) prepared by Reverse Transcription in which RNA (ribonucleic acid) is used as a template is also included in the template DNA. Further, an amplicon which has been prepared by executing PCR on a target sample of DNA profiling and/or a sample obtained from a subject to cancer diagnosis is also included in the template DNA.

A term “stutter” refers to an artifact where the repeat number is increased or reduced as compared with an original sequence thereof upon DNA amplification. Herein, a sequence in which the stutter occurs is referred to as “stutter sequence”, and an amplification product (i.e., amplicon) in which the stutter occurs is referred to as “stutter product”. On the other hand, sometime, an amplification product of a case where an original sequence is correctly amplified is referred to as “True allele”.

### Example Embodiment 1

First, a basic mode of a DNA amplification method of the disclosure is explained as an example embodiment 1. In the DNA amplification method of the example embodiment 1, first, a reaction solution containing a template DNA comprising a microsatellite, primers, a polymerase, and a recombinase is prepared. Then, the prepared reaction solution is subjected to constant temperature incubation so as to amplify a DNA sequence comprising a microsatellite in the template DNA.

As a concrete protocol, a protocol in a commercially available DNA amplification kit may be used, in which RPA: Recombinase Polymerase Amplification is used. As followed, a case is explained as one example, where TwistAmp(R) Basic of TwistDx™ is used for amplifying a DNA sequence comprising a repeat sequence of D1S1656 while adding notes to a protocol disclosed in a manual ver.01 of the kit.

1. Preparing a rehydration solution of each sample as follows.

Primer A (10 μM) 2.40 μl

Primer B (10 μM) 2.40 μl

Rehydration buffer 29.5 μl

Template and dH2O 13.2 μl

(Total Volume 47.5 μl)

Stirring hardly and spinning briefly.

Here, Primer A is a so called forward oligo nucleotide having a sequence, for example, 5′-AAGCCTGTGTTGCTCAAGGGTCAACTGTAT-3′. In addition, Primer B is a so called reverse oligo nucleotide having a sequence and a fluorescent substance (FL: fluorescent), for example, 5′-[FL]-GGATTCTTCAGAGAAATAGAATCACTAGGGAACC-3′.

Such primers are designed based on front and back sequences of a microsatellite (sequences in flanking regions). For example, STRBase discloses a primer set for amplifying a DNA sequence comprising a repeat sequence of D1S1656. Here, primers having 30 to 35 nucleotides and 40 to 60% of GC contents are recommended in TwistAmp (R). When the sequences are re-designed so as to satisfy the recommended condition, the Primers A, B as indicated above will result. Here, Primers A, B are mere one example. In addition, primer design involves consideration in complementarity between forward/reverse oligo nucleotides, dimerization of each primer, and self-annealing (including formation of a hairpin structure, and the like), which are within a technological capacity of a person skilled in the art.

Template is an initial template DNA in DNA amplification as defined in template DNA above, thus may be prepared by using a commercially available nucleic acid extraction kit.

2. Transferring 47.5 μl of a rehydration solution of each sample to a reaction pellet. In addition, up and down-mixing with a pipet until the pellet is entirely resuspended.

Here, the reaction pellet is a microtube containing freeze-dried polymerase and recombinase, thus a reaction system is established by injecting the rehydration solution. The resultant solution having such condition is referred to as “reaction solution” in the application.

3. Supplying 2.5 μl of 280 mM magnesium acetate to each sample followed by well mixing.

Magnesium acetate is a reagent triggering the amplification reaction. In the application, there is also a case where a solution having a condition where the magnesium acetate has been injected is referred to as “reaction solution”.

4. Inserting the tube into a pertinent incubator block (optimally 37 to 42° C.) followed by incubating for 4 minutes.

5. After 4 minutes, taking out the sample from the incubator, mixing by hardly inverting it 8 to 10 times, spinning it and returning the resultant sample to the incubator block.

6. Continuing incubation/detection for total 20 to 40 minutes as a standard.

These steps may be also reworded as “subjecting the reaction solution to constant temperature incubation so as to amplify a DNA sequence comprising the microsatellite in the template DNA” in the application.

Herein, the above protocol may be modified as long as not departing from a concept of the protocol. For example, with respect to mixing, pipetting may be changed to turning over of a tube. In addition, the incubation time period is a mere standard, thus may be extended or shortened. Such modifications in the protocol is also included in the disclosure, and an optimal condition may be determined by executing a pilot test.

The reaction solution containing DNA sequence amplified as described above is subjected to electrophoresis and/or DNA sequence. Herein, the repeat number of a repeat sequence in the amplified DNA sequence is analyzed. That is, as the electrophoresis and the like, any methods may be adopted if the repeat number of the repeat sequence in the amplified DNA sequence can be analyzed. Herein, it is also considered that an amplicon amplified by RPA is purified and subsequently subjected to the electrophoresis.

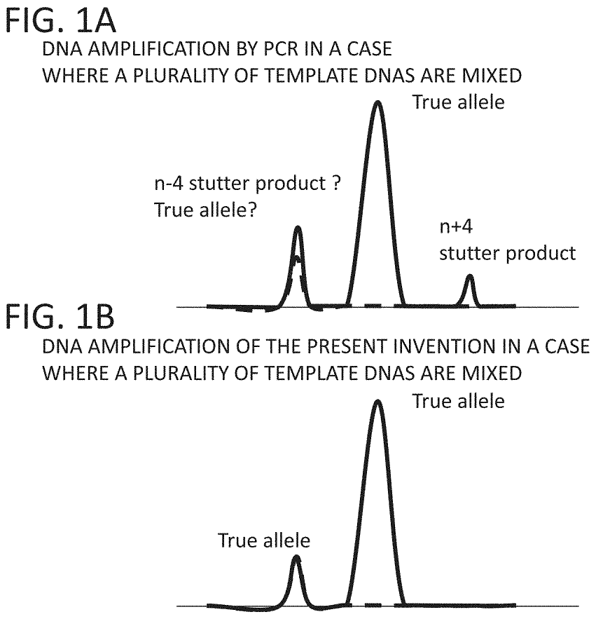

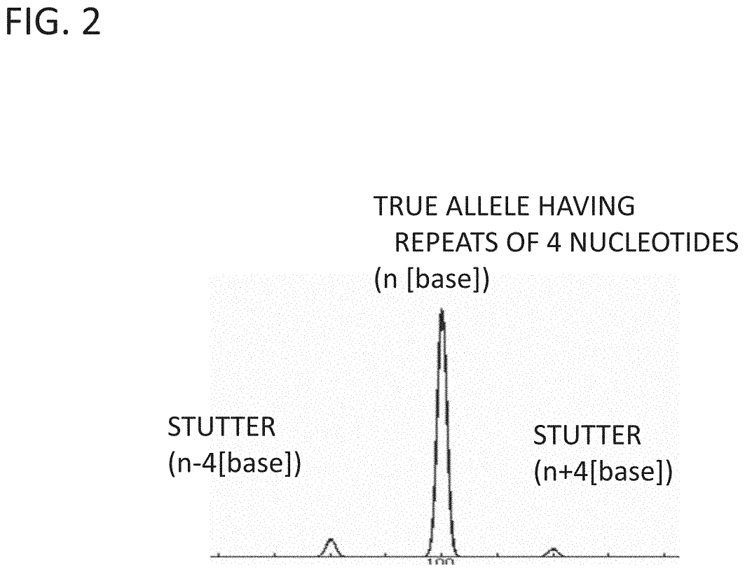

Herein, using a model of waveform obtained by a polyacrylamide gel capillary electrophoresis, an effect of the DNA amplification method of the disclosure is explained. In the capillary electrophoresis, the amplified DNA sequence is applied at the upstream side of a capillary, migrates to the downstream side due to electric current applied to the capillary, and is detected by a fluorescence detection apparatus which monitors the downstream side of the capillary. At that time, a sequence having a shorter nucleotide length is detected in advance of a sequence having a longer nucleotide length due to a molecular sieve effect. FIGS. 1A, 1B and 2 illustrate graphs showing time dependent change in fluorescence intensity detected by the fluorescence detection apparatus. Herein, the graphs of FIGS. 1A and 1B are a partial modification of a model disclosed in NIST, respectively (FIG. 2 of the application: https://strbase.NIST. gov/pub_pres/Aponte-AAFS2016-sequence-based-STR-stutter.pdf).

Like as the model disclosed in NIST (FIG. 2 of the application), in a case where a single template DNA is amplified by PCR, peaks of stutter products appear before and after a peak of a correctly amplified DNA sequence (True allele). Herein, in a case of multiple template DNAs, there is a case where products derived from a plurality of template DNA overlap one another on the same peak. Such state may provide a critical defect in the DNA profiling and cancer diagnosis. In addition, if a plurality of template DNAs is included at different ratios, there is also a case where the stutter product and the True allele cannot be discriminated.

As one example, a case is discussed, where DNA profiling is executed for a purpose of specifying an offender from bloodstain left in a crime scene. It is supposed that the bloodstain left in a crime scene includes a large amount of DNA of a victim and a small amount of offender's DNA. Herein, a premise is made that the victim has homo isoallele A and the offender has homo isoallele B. Here, a premise is also made that the isoallele B has the same nucleotide length with n-4 stutter product of the isoallele A. When the bloodstain is subjected to PCR and then amplified DNA sequences are detected, a graph shown in FIG. 1A is obtained. In FIGS. 1A and 1B, a solid line is a model of an actual waveform and a dotted line is a waveform model of a case where the template DNA includes only the offender's DNA. Herein, the n-4 stutter product derived from victim's DNA overlaps with True allele derived from the offender's DNA on the same peak. Therefore, not only profiling of the offender cannot be realized, but also it cannot be determined whether or not bloodstain left in the crime scene includes the offender's DNA.

On the other hand, in the DNA amplification method of the disclosure, a graph indicated in FIG. 1B is obtained since occurrence of stutter is prevented or suppressed. That is, there is no occurrence of overlapping of n-4 stutter product derived from the victim's DNA and True allele derived from the offender's DNA. Therefore, based on peak height, a higher peak can be regarded as a True allele derived from the victim's DNA and a lower peak can be regarded as a True allele derived from the offender's DNA. That is, according to the DNA amplification method of the disclosure, profiling of the offender can be performed.

[Technical Significance of the DNA Amplification Method of the Disclosure]

As described in the above protocol, the DNA amplification method of the disclosure is one along a protocol of a general recombinase polymerase amplification (RPA), and includes a feature that a template DNA comprising a microsatellite is used. The technical significance of the feature that “a template DNA comprising a microsatellite is used” is explained as follows.

DNA amplification by RPA is a technology which has been developed in about 2006, thus has a shorter history as compared with PCR (Polymerase Chain Reaction). Therefore, in the DNA profiling and cancer diagnosis in which reliability is important, DNA amplification by PCR is adopted. In addition, PCR has an advantage that the primers are shorter than those of RPA.

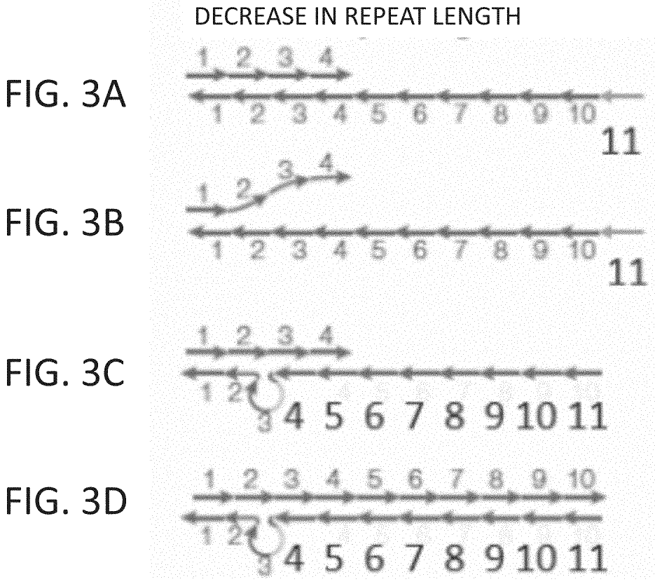

However, in the DNA amplification by PCR, the stutter occurs and provides a negative effect on the accuracy in the DNA profiling and cancer diagnosis. Although there are several theories for the mechanism of occurrence of the stutter(s), a model illustrated in FIGS. 3A to 3D is explained when referring to, for example, Nature Reviews Genetics, volume 5, pages 435-445 (2004). FIGS. 3A to 3D show a partially modified figure of the review.

In the DNA amplification by PCR, a double-strand DNA (dsDNA) is denatured into single-strand DNAs (ssDNAs), and primers are annealed, subsequently extension reaction with a polymerase is performed (FIG. 3A). Herein, an extended chain and a template chain are bonded by transient hydrogen bond, and the extended chain and the template chain repeat cleavage and re-binding (FIG. 3B). At that time, there is a case where the template chain is re-bonded with the extended chain while forming a bulge structure (FIG. 3C). When the extension reaction is proceeded under such state, a stutter amplicon having a reduced repeat number is generated (FIG. 3D).

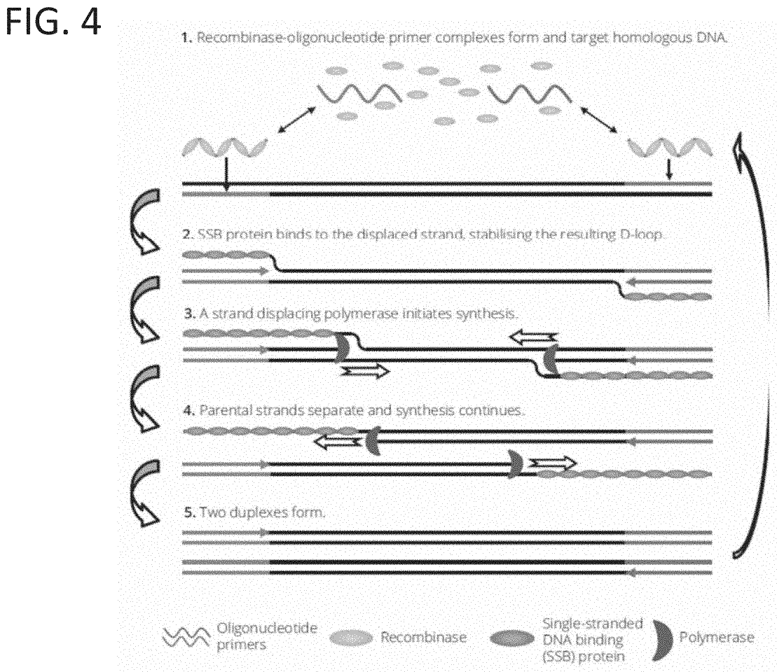

According to such mechanism of occurrence of the stutter, the inventor of the application was motivated to adopt DNA amplification by RPA for a purpose of prevention or suppress of occurrence of the stutter. Here, DNA amplification by RPA is explained as a model shown in FIG. 4. It is a significant point focused by the inventor of the application, in the DNA amplification by RPA, the extension reaction by the polymerase is executed while maintaining double-strand DNA as much as possible. Here, FIG. 4 is a model disclosed in Combined Instruction Manual for TwistAmp (R) Liquid DNA Amplification Kits of TwistDx (TM).

In summary, the inventors of the application made the disclosure along the following approach.

(1) Reaching a problem that occurrence of stutter is to be prevented or suppressed.

(2) Focusing on formation of a bulge structure due to transient cleavage and re-binding between an extended chain and a template chain as a cause of the occurrence of the stutter.

(3) Finding a characteristic of RPA that, transient cleavage and re-binding between the extended chain and the template chain hardly occur, and thus adopting it to DNA amplification for a template DNA comprising a microsatellite.

That is, a person skilled in the art cannot reach the disclosure without such approach. In other words, such approach is explained as one technical significance of the disclosure.

### Example Embodiment 2

Next, an application mode of the DNA amplification method of the example embodiment 1 is explained as an example embodiment 2.

The disclosure may be provided as a DNA amplification kit. The DNA amplification kit comprises: a polymerase and a recombinase which are prepared in a reaction solution for amplifying a DNA sequence comprising a microsatellite, and primers for amplifying the DNA sequence.

For example, a DNA amplification kit used for DNA profiling, such as paternity test, comprises a microtube for each of microsatellites such as D1S1656 and CSF1PO. The microtube comprises, in addition to the polymerase and the recombinase, primers for each of the microsatellites in order to amplify a template DNA comprising the microsatellites. Such primers may be prepared while referring to STRBase of NIST, as described above. Such DNA amplification kit is provided as a kit specialized to DNA profiling, and is different from a commercially available DNA amplification kit which uses RPA as described above. The same is applied to a case where it is provided as a kit specialized to cancer diagnosis.

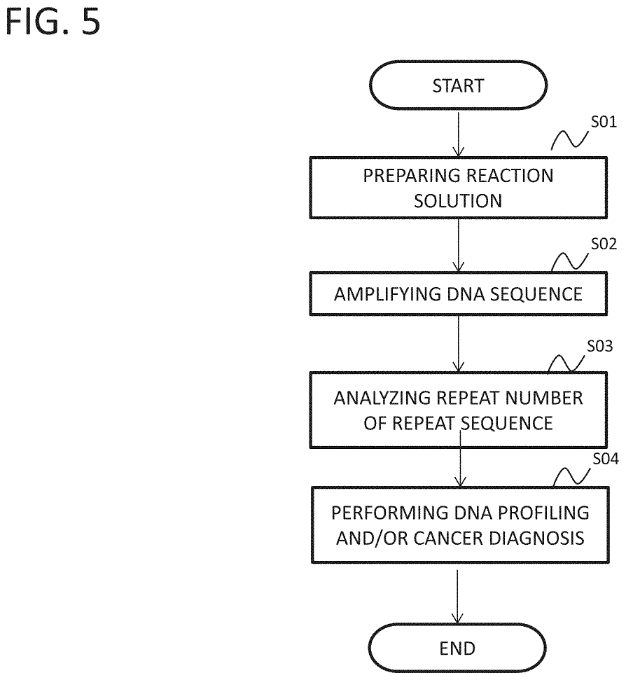

The disclosure is provided as a profiling/diagnosis method for performing DNA profiling and/or cancer diagnosis. That is, as illustrated in FIG. 5, the profiling/diagnosis method of the disclosure comprises: a reaction solution preparation step (step S01), an amplification step (step S02), an analysis step (step S03) and a profiling/diagnosis step (step S04). Concretely, in step S01, a reaction solution containing a template DNA comprising a microsatellite, primers, a polymerase, and a recombinase is prepared. Next, in step S02, the reaction solution is subjected to constant temperature incubation so as to amplify a DNA sequence comprising the microsatellite in the template DNA. Next, in step S03, the reaction solution after an amplification reaction is subjected to electrophoresis and/or DNA sequence so as to analyze the repeat number of a repeat sequence. And then, in step S04, DNA profiling and/or cancer diagnosis is performed using an analysis result.

### Example Embodiment 3

Next, a modified mode of the DNA amplification method of the example embodiment 1 is explained as an example embodiment 3.

RPA is a reaction system of constant temperature incubation, thus involving no concept of “cycle”, as different from PCR. Here, it is considered that the amount of the primers is set as a bottleneck in order to prevent unnecessary progress of DNA amplification reaction (in other words, unnecessary proliferation of an amplicon). That is, a protocol is so designed that the primers are to be exhausted, thereby DNA amplification reaction may be stopped due to exhaustion of the primers.

Herein, for example, if the protocol is so designed that the primers are stepwisely supplied, a cycle from supplementation of the primers once to exhaustion of the primers would be regarded as a pseudo one-cycle.

In addition, if the protocol is so designed that the primers are prepared in a state of a double-strand DNA and the primers are to be denatured into single-strand DNAs by temporal heating, one heating step may be regarded as one-cycle. That is, the primers do not serve a function as the primers under the state of double-strand DNA where the primers bond with their complementary chains, thus a state where the primers exist in the double-strand DNA may be said as a pseudo exhaustion state of the primers. Here, when the primers of the double-strand DNA are denatured into single-strand DNAs by heating, the primer are made capable of annealing with a template DNA, and the exhaustion state of the primers is cancelled. Note, the temperature of the temporal heating may be defined as a temperature at which although the primers are denatured into the single-strand DNAs, the template DNA is, at least partially, maintained in the state of double-strand DNA, and as a temperature at which the polymerase and the recombinase are not inactivated. The temperature at which the primers are denatured into single-strand DNAs may be designed by modifying primer sequence (modification according to Tm (Melting Temperature) value), and by introducing a mismatch sequence(s) into complementary chains. Such design of the primers and the complementary chains may be adopted as far as occurrence of the stutter would be prevented or suppressed finally.

### Example Embodiment 4

Next, an example embodiment is explained as an example embodiment 4, where PCR is performed so as to prepare a template DNA before the recombinase polymerase amplification in the DNA amplification method of the example embodiment 1.

As described above, when compared with PCR, the recombinase polymerase amplification (RPA) involves a stringent condition in the design of the primers and the like even in a case where a commercially available DNA amplification kit is used. That is, RPA can be said as a troublesome (delicate) DNA amplification method as compared with PCR. Here, in a case where a sample has a bad quality, such as bloodstain left in a crime scene and the like, even the DNA amplification using PCR encounters a difficulty, thus RPA encounters a further difficulty.

Therefore, it is considered that difficulty in the DNA amplification is reduced by combining PCR with RPA. That is, in first, sample quality is improved by PCR of several cycles. Next, RPA is performed using the DNA amplified by PCR (i.e., amplicon) as a template DNA. Thereby, the difficulty in the DNA amplification may be reduced.

Here, in the DNA amplification where PCR and RPA are combined, PCR performed before RPA may be expressed as “pre-PCR”, “pre-amplification” and the like, which is regarded as a step of preparing the template DNA to be subjected to RPA. Although there is a possibility that stutter occurs in the pre-PCR, occurrence of the stutter may be suppressed by combining PCR and RPA as compared with DNA amplification using PCR only. That is, for example, as compared with DNA amplification by PCR of 30 cycles, DNA amplification where PCR of 4 cycles and RPA are combined may suppress occurrence of the stutter since DNA amplification corresponding to 5th and following cycles in PCR is replaced with RPA.

Here, the cycle number of the pre-PCR may be designed while taking into account the quality of original sample, the quality of the primers and the like, but not limited to 4 cycles. In addition, DNA amplified by PCR may be purified so as to further improve the quality of the sample (template DNA).

### EXAMPLES

An experiment of amplifying a DNA sequence was actually performed using the DNA amplification method of the disclosure. Material and method, result, discussion of the experiment are described as follows. In addition, a comparative experimental result of a case where DNA amplification by PCR is performed is described as a comparative example.

(Material and Method)

[Reagents]

| Type | Kit | Reagent name | Density | Manufacturer | Ref | Lot |
|---|---|---|---|---|---|---|
| template | | 2800M Control DNA | 10 ng/uL | Promega | AX387X | Lot.0000186886 |
| DNA | | | | | | |
| PCR | | 5x PrimeSTAR® GXL Buffer Mg2+ plus | 5x | TaKaRa | SD1967 | Lot.2301A |
| | | PrimeSTAR® GXL DNA Polymerase | 1.25 U/uL | TaKaRa | R050A | Lot.N2001BA |
| | | dNTP Mixture | 2.5 mM each | TaKaRa | 4030 | Lot.BJ6401A |
| RPA | TwistAmp® Basic | | | TwistDx | TABAS03KIT | |
| | | Primer Free Rehydration buffer | | | | Lot. 108769 |
| | | Magnesium Acetate (MgOAc) | 280 mM | | | Lot. 108765 |

| | |
|---|---|
| primer | Forward Primer (23mer) D5-PP16-FFAM: |
| | FFAM-GGTGATTTTCCTCTTTGGTATCC |
| | Reverse Primer (26mer) D5-PP16-R: |
| | AGCCACAGTTTACAACATTTGTATCT |

| | | | | |
|---|---|---|---|---|
| purification | NucleoSpin® Gel and PCR Clean-up | MACHEREY-NAGEL | 74609 | Lot. 1705/001 |

[Apparatuses]

| Usage | Name of machine | Manufacturer |
|---|---|---|
| PCR, RPA | Mastercycler ® nexus | eppendorf |
| | C1000 ™ Thermal Cycler | BIO-RAD |
| | CFX96 ™ Real-Time System | BIO-RAD |
| electrophoresis | 3730XL | ThermoFisher |

[Protocol of Comparative Example (Only PCR)]

Performed were 30 rounds of PCR cycle including a denaturing step at 98° C., a primer annealing step at 60° C., and an extension step at 68° C.

| PCR protocol (25 uL reaction) | | | | final concentration | |
|---|---|---|---|---|---|
| 5 | uL | 5x Prime STAR GXL Buffer | | 1 | x |
| 2 | uL | 2.5 mM dNTP Mixture | | 0.2 | mM |
| 2 | uL | 2.5 uM Forward Primer (D5-PP16-FFAM) | | 0.2 | uM |
| 2 | uL | 2.5 uM Reverse Primer (D5-PP16-R) | | 0.2 | uM |
| 0.5 | uL | 10 ng/uL template DNA, 2800M | | 0.2 | ng/uL |
| 0.5 | uL | PrimeSTAR GXL DNA Polymerase | | 0.02 | x |
| 13 | uL | Distilled Water | | | |
| 25 | uL | | | | |
| | | 98′C, 10 sec | 30 cyc. | | |
| | | 60′C, 15 sec 3 | | | |
| | | 68′C, 60 sec | | | |
| | | 4′C | | | |

[Protocol of Example (PCR+RPA)]

First, performed were 4 rounds of PCR cycle including a denaturing step at 98° C., a primer annealing step at 60° C. and an extension step at 68° C.

| PCR protocol | | | final concentration | |
|---|---|---|---|---|
| 5 | uL | 5x Prime STAR GXL Buffer | 1 | x |
| 2 | uL | 2.5 mM dNTP Mixture | 0.2 | mM |
| 2 | uL | 2.5 uM Forward Primer (D5-PP16-FFAM) | 0.2 | uM |
| 2 | uL | 2.5 uM Reverse Primer (D5-PP16-R) | 0.2 | uM |
| 0.5 | uL | 10 ng/uL template DNA, 2800M | 0.2 | ng/uL |
| 0.5 | uL | PrimeSTAR GXL DNA Polymerase | 0.02 | x |
| 13 | uL | Distilled Water | | |
| 25 | uL | | | |

| | |
|---|---|
| 98′C, 10 sec | 4 cyc. |
| 60′C, 15 sec | |
| 68′C, 60 sec | |
| 4′C | |

Next, PCR amplicon generated by the above PCR was purified according to a protocol of NucleoSpin.

PCR-amplicon purification
PCR amplicon subjected to pufirication = 25 uL
elution volume = 25 uL

Next, RPA was performed using the purified PCR amplicon as a template DNA.

| RPA protocol | | | final concentration | |
|---|---|---|---|---|
| 3.2 | uL | DW | 0.48 | uM |
| 29.5 | uL | Primer Free Rehydration buffer | 0.48 | uM |
| 2.4 | uL | 10 uM Forward Primer (D5-PP16-FFAM) | | |
| 2.4 | uL | 10 uM Reverse Primer (D5-PP16-R) | | |
| 10 | uL | purified PCR amplicon | | |
| 47.5 | uL | | | |
| ↓ | | | | |

Entire solution was injected into reaction tube of the kit.

| ↓ | | |
|---|---|---|
| 2.5 | uL | 280 mM MgOAc |
| 50 | uL | |

39′C, 20 min

Then, the RPA amplicon generated by the above RPA was purified according to a protocol of NucleoSpin.

RPA-amplicon purification
RPA amplicon subjected to purification = 25 uL
elution volume = 25 uL

(Results)

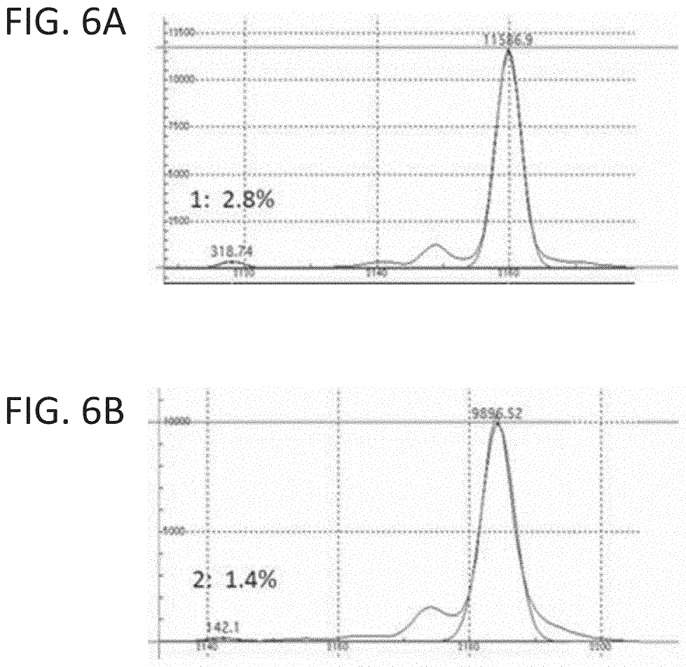

FIG. 6A shows an electrophoresis result of the comparative example (PCR only). In a case of DNA amplification using PCR only provided a 2.8% stutter peak relative to a True allele peak.

FIG. 6B shows an electrophoresis result of the Example (PCR+RPA). In a case of DNA amplification using the combination of PCR and RPA provided a 1.4 stutter peak relative to a True allele peak.

(Discussion)

The DNA amplification by PCR+RPA of the Example provided the 1.4% stutter peak, which is significantly smaller than the 2.8% stutter peak of the DNA amplification by PCR only of the comparative example. It cannot be determined whether the 1.4% stutter peak has been provided in PCR of 4 cycles of the Example or in the subsequent RPA. However, it can be said that DNA amplification by RPA has reduced occurrence of the stutter as compared with 5th and following cycles in PCR of the comparative example.

In addition, the primers used in the above Example have 23 mer (Forward) and 26 mer (Reverse), which are shorter than those having 30 to 35 nucleotide length recommended in a general RPA kit. Therefore, it is supposed that the DNA amplification may be performed without the pre-PCR if primers having 30 to 35 nucleotide length are used.

Further, although purification of PCR amplicon was performed in the above Example, such purification was not an essential step. For example, the recombinase may be supplied to the reaction system at the stage of PCR so that PCR and RPA are performed as one successive operation. Here, although there is a possibility that a slight correction would be required in a buffer composition and the like, such correction seems to be within a technological capacity of a person skilled in the art. Further, the recombinase may be directly supplied to the reaction system after PCR.

A part or entire of the exemplary embodiments may be described as follows, but not limited thereto.

(Mode 1)

A DNA amplification method, comprising:

a step of preparing a reaction solution containing a template DNA comprising a microsatellite, primers, a polymerase, and a recombinase, and

a step of subjecting the reaction solution to constant temperature incubation so as to amplify a DNA sequence comprising the microsatellite in the template DNA.

(Mode 2)

The DNA amplification method according to Mode 1, wherein the template DNA is a target sample of DNA profiling and/or a sample obtained from a subject to cancer diagnosis.

(Mode 3)

The DNA amplification method according to Mode 2, wherein

the target sample of DNA profiling is a sample comprising DNAs from a plurality of persons.

(Mode 4)

The DNA amplification method according to Mode 3, wherein

the target sample of DNA profiling is a sample containing DNAs of a plurality of persons at different ratios.

(Mode 5)

The DNA amplification method according to any one of Modes 1 to 4, further comprising

a step of preparing the template DNA by PCR.

(Mode 6)

A DNA amplification kit, comprising:

a polymerase and a recombinase which are prepared in a reaction solution for amplifying a DNA sequence comprising a microsatellite, and

primers for amplifying the DNA sequence.

(Mode 7)

A profiling/diagnosis method, comprising:

a step of amplifying a DNA sequence comprising a microsatellite according to the DNA amplification method of Mode 1,

a step of subjecting a reaction solution after an amplification reaction to electrophoresis and/or DNA sequence so as to analyze repeat number of a repeat sequence, and

a step of executing DNA profiling and/or cancer diagnosis using an analysis result by analysis.

Herein, disclosure of the above Patent Literature is incorporated in the application. The exemplary embodiment(s) or example(s) may be modified or adjusted within the scope of the entire disclosure of the disclosure, inclusive of claims, based on the fundamental technical concept of the invention. In addition, a variety of combinations or selections (including partial selection or non-selection) of disclosed variety elements (each element in each claim, each element in each example embodiment or each example, etc.) may be made within the claims of the disclosure. That is, the disclosure, of course, may cover a variety of modifications or corrections that may be made by those skilled in the art in accordance with the entire disclosure of the disclosure, inclusive of claims, and the technical concept of the disclosure. Note, it is regarded as being included in matters disclosed in the application to combine a part or entire of the cited Patent Literature(s) with the description of the application.

What is claimed is:

1. A DNA amplification method, comprising:
preparing a reaction solution containing a template DNA comprising a microsatellite, primers, a polymerase, and a recombinase, and
subjecting the reaction solution to constant temperature incubation so as to amplify a DNA sequence comprising the microsatellite in the template DNA.

2. The DNA amplification method according to claim 1, wherein
the template DNA is a target sample of DNA profiling and/or a sample obtained from a subject to cancer diagnosis.

3. The DNA amplification method according to claim 2, wherein
the target sample of DNA profiling is a sample comprising DNAs from a plurality of persons.

4. The DNA amplification method according to claim 3, wherein
the target sample of DNA profiling is a sample containing DNAs of a plurality of persons at different ratios.

5. The DNA amplification method according to claim 1, further comprising
preparing the template DNA by PCR.

6. A DNA amplification kit, comprising:
a polymerase and a recombinase which are prepared in a reaction solution for amplifying a DNA sequence comprising a microsatellite, and
primers for amplifying the DNA sequence.

7. A profiling/diagnosis method, comprising:
amplifying a DNA sequence comprising a microsatellite according to the DNA amplification method of claim 1,
subjecting a reaction solution after an amplification reaction to electrophoresis and/or DNA sequence so as to analyze repeat number of a repeat sequence, and
executing DNA profiling and/or cancer diagnosis using an analysis result by analysis.

8. The DNA amplification method according to claim 2, further comprising
preparing the template DNA by PCR.

9. The DNA amplification method according to claim 3, further comprising
preparing the template DNA by PCR.

10. The DNA amplification method according to claim 4, further comprising
preparing the template DNA by PCR.
